(19) United States
(12) Patent Application Publication
(10) Pub. No.: US 2019/0151525 A1
(43) Pub. Date: May 23, 2019

(54) DEVICE TO SECURE A CATHETER PORT

(71) Applicant: Norris Alaiasa, Laie, HI (US)

(72) Inventor: Norris Alaiasa, Laie, HI (US)

(21) Appl. No.: 15/820,845

(22) Filed: Nov. 22, 2017

(51) Int. Cl. A61M 1/28

(52) U.S. Cl. CPC A61M 1/285; A61F 13/0206

(57) ABSTRACT

A device to hold peritoneal dialysis catheters with exit wand and folding line also paper or elastic loop also a non-stick peel off tab sterilized gauze and adhesive.

### BACKGROUND OF THE INVENTION

### Field of the Invention

The present invention is generally in the medical industry. More specifically, the present invention is a device to hold peritoneal dialysis catheters.

### Background of the Invention

The present invention is a PD (peritoneal dialysis) anchor that has a bandage, gauze and loops. The main purpose of the PO anchor is to provide consumers with a safe and easy way to secure PD catheters. Many who frequently go through dialysis procedures often have catheters placed in their abdomen along with an outlet tube so that the medication is passed through; however, caring for the exit wound and securing the catheter in place can be difficult as it has to be kept clean and away from clothing as well as other items that can pull or tug on it. Moreover, common methods of securing PD catheters include the use of adhesive strips of tape which are prone to coming off and are unsanitary. Ingenious, practical and useful, the PD Anchor is an innovative alternative to conventional methods of protecting exit wounds while securing the catheter in place. This cutting-edge product is comprised of a 2-piece bandage measuring 3″×6″ with incorporated gauze and (2) loops. Easy to use, simply place the afforded bandage directly over the catheter/exit wound; the built-in 2″×2″ gauze is located under the flexible adhesive bandage to securely hold the catheters in position. The PD anchor is essential for the everyday consumer and may be readily available at hospitals, clinics, pharmacies, and retail department stores.

The PD anchor is the only product of its kind that features a two part bandage through which the excess tubing can be threaded and placed in a safe and secure manner via the afforded loops in order to keep their catheter anchored and close to their bodies. The PD anchor is uniquely designed to be manufactured with medical grade materials for durability and is also packaged in a box of (50) to meet any consumer need.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a back view of a device to hold peritoneal dialysis catheters, in accordance with one embodiment of the present invention.

FIG. 2 illustrates a front view of a device to hold peritoneal dialysis catheters, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

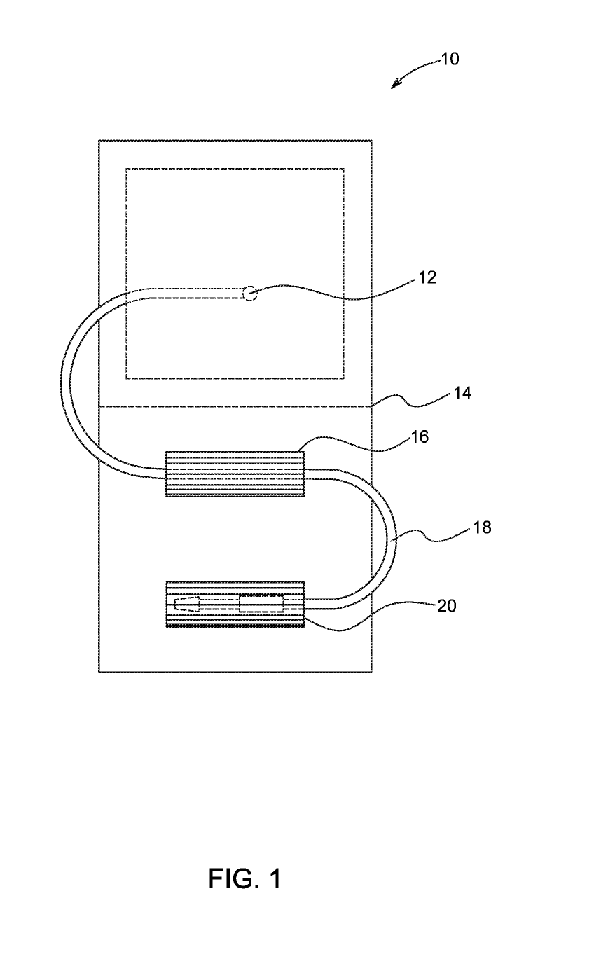

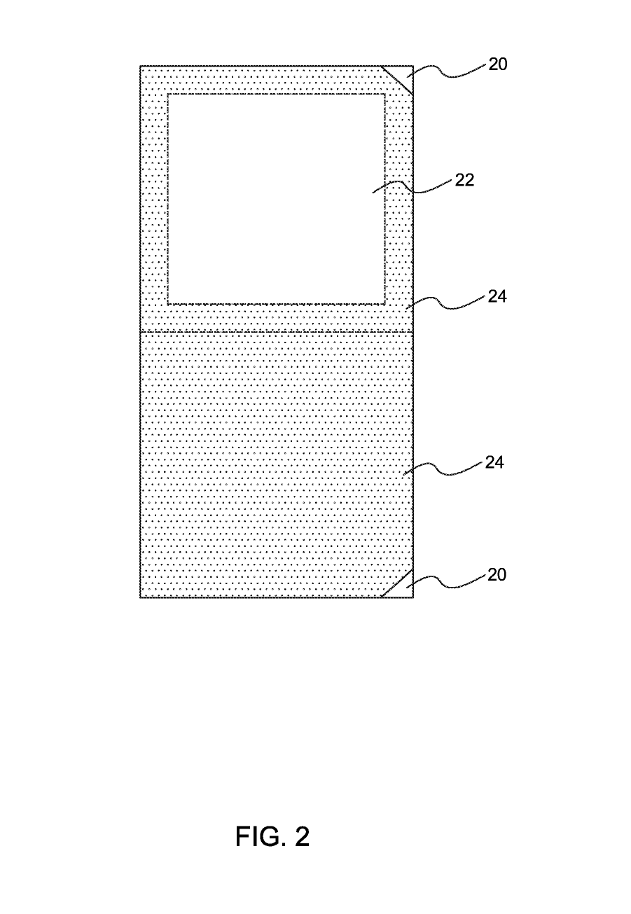

As shown in FIG. 1 of the present invention is a device to hold peritoneal dialysis catheters 10 with exit wand 12 and folding line 14 also paper or elastic loop 16-hose 18. As shown in FIG. 2 of the present invention is a non-stick peel off tab 20 sterilized gauze 22 and adhesive 24.

While the present invention has been related in terms of the foregoing embodiments those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A peritoneal dialysis anchor comprising:
an exit wand at the end of a hose,
a folding line on a bandage, with a paper or elastic loop attached to the bandage for the hose to pass though, and
at least one non-stick peel off tab, a sterilized gauze around the exit wand and adhesive to hold the bandage together when folded.

2. A peritoneal dialysis anchor according to claim 1, wherein the bandage is three inches by six inches.

3. A peritoneal dialysis anchor according to claim 1, wherein the gauze is two inches by two inches
